(19) United States
(12) Patent Application Publication
(10) Pub. No.: US 2024/0127908 A1
(43) Pub. Date: Apr. 18, 2024

(54) METHOD AND APPARATUS FOR THE DISCOVERY OF TARGET ANTIGENS FOR CHIMERIC ANTIGEN RECEPTORS

(71) Applicant: KOREA ADVANCED INSTITUTE OF SCIENCE AND TECHNOLOGY, Daejeon (KR)

(72) Inventors: Jung Kyoon CHOI, Daejeon (KR); Joon Ha KWON, Daejeon (KR)

(21) Appl. No.: 18/142,958

(22) Filed: Jan. 3, 2024

Related U.S. Application Data
(63) Continuation-in-part of application No. PCT/KR2021/016455, filed on Nov. 11, 2021.

(30) Foreign Application Priority Data
Nov. 11, 2020 (KR) 10-2020-0149990

(51) Int. Cl. G16B 40/00; G16B 25/10

(52) U.S. Cl. CPC G16B 40/00; G16B 25/10

(57) ABSTRACT

A method for discovering a target antigen for a chimeric antigen receptor comprises the steps of: obtaining, by an analysis apparatus, expression information of genes of tumor cells in single cell units; extracting, by the analysis apparatus, from among the genes, genes of candidate antigens having an expression level of a reference value or more; determining, by the analysis apparatus, all possible gene combinations that can be constituted by the genes of the candidate antigens; and determining, by the analysis apparatus, a target gene combination in which at least one gene included therein is expressed, among the gene combinations.

### CROSS-REFERENCE TO RELATED APPLICATION

This application is a continuation-in-part application of PCT Application No. PCT/KR2021/016455, filed on Nov. 11, 2021, which claims the benefit of and priority to Korean Patent Application No. 10-2020-0149990, filed on Nov. 11, 2020. The entire disclosures of the applications identified in this paragraph are incorporated herein by references.

### FIELD

The technology described below relates to a method of discovering an antigen for a chimeric antigen receptor (CAR).

### BACKGROUND

Immune cell therapy for cancer is a therapeutic method of removing tumor cells using ex vivo cultured T cells. A CAR is a binding site recognizing an antigen that is designed to bind to an active domain in T cells. CAR-T is T cell therapy that is applied to T cells. On the other hand, CAR-NK is cell therapy that is applied to natural killer (NK) cells.

For CAR-T and CAR-NK, effective target antigen discovery is very important. In the target antigen discovery, coverage which indicates whether CAR-T and CAR-NK act on a large number of tumor cells, and specificity which indicates whether cell toxicity applied to normal cells is minimized and they act only on tumor cells are critical indicators.

### SUMMARY

One aspect of the technology described below provides a method of discovering a target antigen for a CAR, which includes acquiring expression information of genes of tumor cells in a single cell unit by an analysis apparatus, extracting genes of candidate antigens having an expression level of a reference value or higher from the genes by the analysis apparatus, determining gene combinations which can consist of the genes of the candidate antigens by the analysis apparatus, determining a target gene combination in which at least one gene included in the combination is expressed among the gene combinations by the analysis apparatus, and determining at least one of the target gene compositions as a gene combination for a target antigen by the analysis apparatus.

Another aspect of the technology described below provides an analysis apparatus for CAR design, which includes an input device for receiving expression data of genes in tumor cells of a sample in a single cell unit, a storage device for storing a program that determines an antigen recognized by a CAR using the expression information of genes in tumor cells, and an arithmetic device for determining a target gene combination or target gene for a target antigen of tumor cells in the sample recognized by the CAR from gene combinations that can consist of a plurality of genes among the genes of the candidate antigens determined from the expression data of the genes.

### BRIEF DESCRIPTION OF THE DRAWINGS

FIGS. 1A, 1B, 1C and 1D illustrate an example of antigen expression patterns in single cells and a cell population.

FIG. 2 illustrates an example of a system for discovering a target antigen for a chimeric antigen receptor (CAR).

FIG. 3 illustrates one example of a flowchart illustrating a process of discovering a target antigen for a CAR.

FIG. 4 illustrates another example of a flowchart illustrating a process of discovering a target antigen for a CAR.

FIG. 5 illustrates still another example of a flowchart illustrating a process of discovering a target antigen for a CAR.

FIG. 6 illustrates an example of a process of discovering a target antigen by an analysis apparatus.

FIGS. 7A and 7B illustrate an experimental result of the gene expression patterns of tumor cells and normal cells in a colorectal cancer sample and a lung cancer sample.

FIGS. 8A and 8B illustrate an experimental result for a gene combination in which one or more genes are expressed in a lung cancer sample.

FIGS. 9A and 9B illustrate an experimental result for a gene combination in which one or more genes are expressed in a colorectal cancer sample.

FIGS. 10A and 10B illustrate an experimental result for a gene combination in which two or more genes are expressed in a lung cancer sample.

FIGS. 11A and 11B illustrate an experimental result for a gene combination in which two or more genes are expressed in a colorectal cancer sample.

FIGS. 12A and 12B illustrate an experimental result for a tumor cell-specific gene combination in a lung cancer sample.

FIGS. 13A and 13B illustrate an experimental result for a tumor cell-specific gene combination in a colorectal cancer sample.

FIG. 14 illustrates an example of an analysis apparatus for discovering an antigen for a CAR.

FIG. 15 illustrates an example of the process of identifying a gene combination that discriminates tumor cells and normal cells using large-scale single cell transcriptome data.

FIGS. 16A and 16B illustrate a result of comparing patterns of RNA expression and protein expression against an antigen of ovarian cancer cells.

FIG. 17 illustrates another example of a process of discovering a target antigen for a CAR.

### DETAILED DESCRIPTION

The technology described below may have various modifications and various examples, and thus specific examples are illustrated in the drawings and described in detail in the detailed description. However, it should be understood that the technology described below is not limited to specific embodiments, and includes all modifications, equivalents or alternatives within the spirit and technical scope of the technology described below.

The terms “first,” “second,” “A,” and “B” may be used to describe various components, but the components should not be limited by these terms. The terms are used only to distinguish one component from another component. For example, without departing from the scope of rights of the technology described below, a first component may be referred to as a second component, and similarly, a second component may also be referred to as a first component. The term “and/or” encompasses a combination of a plurality of related items described herein, or any one of a plurality of the related items described herein.

It should be understood that singular expressions used herein include plural expressions unless clearly interpreted otherwise in the context. In the specification, it should be understood that the term “include” or “have” is intended to indicate the presence of a characteristic, a number, a step, an action, a component or a part, which is described, or a combination thereof, but does not preclude the possibility of the presence or addition of one or more other characteristics, numbers, steps, actions, components, parts or a combination thereof.

Prior to the detailed description of the drawings, it should be clarified that the division of components in the specification is merely the classification of components depending on the main function of each component. That is, two or more components to be described below may be combined into one component, or divided into two or more segments by subdivided function. In addition, it is obvious that each component to be described below may additionally perform some or all of the functions of the functions of other components, in addition to its main function, or some of the main functions of the respective components may be exclusively performed by other components.

In addition, during the accomplishment of a method or an operating method, procedures comprising the method may be performed in a different order from the specified order unless clearly and particularly stated otherwise in the context. That is, the procedures may be performed in the same order as specified, performed substantially simultaneously, or performed in a reverse order.

Terms used below will be described.

An antigen is a material that induces an immune response.

Hereinafter, an antigen is a site in tumor cells, which is recognized by a chimeric antigen receptor (CAR). Antigens may be tumor-specific antigens (TSAs) which are not expressed in normal cells but expressed in tumor cells, or tumor-associated antigens (TAAs) which are expressed in normal cells and also shown at a particularly high frequency in cancer cells due to an antigenic change in the cancerization of cells, but the present invention is not limited thereto.

A CAR mainly uses a single-chain variable fragment (scFv). Accordingly, an antigen may include a protein expressed on the surface of a tumor cell, a sugar chain, and a sugar lipid.

A target antigen described below is defined as a target that is recognized by a CAR for tumor cell death. The technology described below is to discover a target antigen.

A sample is a single cell or multiple cells, a cell fragment, or a body fluid taken from a subject to be analyzed.

Genetic data or genetic information is genetic information produced by analyzing a sample. For example, genetic data may include deoxyribonucleic acid (DNA) and ribonucleic acid (RNA), which are obtained from cells, tissue, etc., a base sequence, genetic expression data, genetic standard genetic data obtained from a protein, genetic variation with standard genetic data, DNA methylation, etc., which are obtained from a protein. Genetic data of cells described below is information related to an antigen mainly expressed on a cell surface. Accordingly, genetic data may include information such as a genome, a transcriptome, and a proteome. That is, genetic data is data from which the degree of specific gene expression (particularly, antigen expression on a cell surface) can be confirmed regardless of its type. Genetic data is assumed to be digital data that can be processed by a computer apparatus.

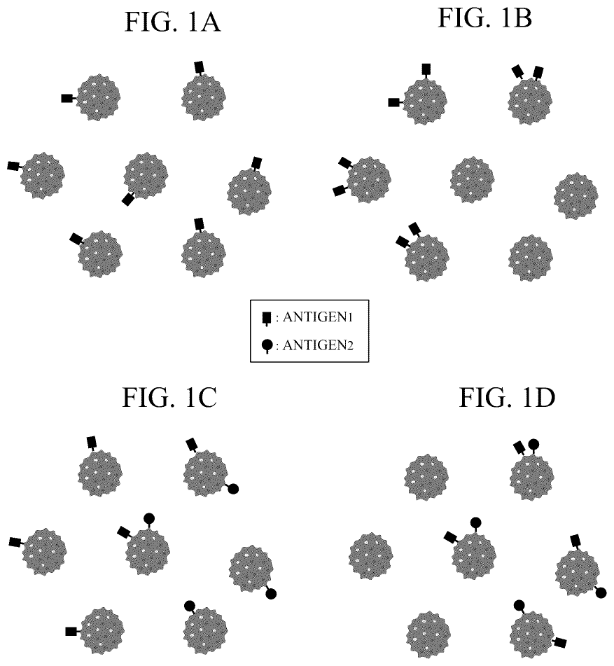

FIGS. 1A-1D illustrate an example of antigen expression patterns in single cells and a cell population.

FIGS. 1A and 1B illustrate the state in which a single antigen is expressed in tumor cells. In FIGS. 1A and 1B, the single antigen is antigen 1. FIG. 1A is an example of expression observed at a single cell level, and FIG. 1B is an example of expression observed in a cell population. Referring to FIG. 1A, antigen 1 has 100% coverage for a single cell. Referring to FIG. 1B, the antigen 1 has 4/7 coverage for a cell population. Accordingly, when CAR-T is designed as a CAR recognizing the antigen 1 confirmed to be expressed based on the result of FIG. 1B, the corresponding CAR-T attacks only a specific clone in tumor cells and prevents other clones in which the corresponding antigen is not expressed from being removed. The same is true of CART-NK.

FIGS. 1C and 1D illustrate the state in which a plurality of antigens are expressed in tumor cells. In FIGS. 1C and 1D, the plurality of antigens include antigen 1 and antigen 2. FIG. 1C is an example of observing expression to a single cell level, and FIG. 1D is an example of observing expression in a cell population. Referring to FIG. 1C, the antigens 1 and 2 have 100% coverage for a single cell. Referring to FIG. 1D, the antigens 1 and 2 have 4/7 coverage for a cell population. Accordingly, when CAR-T is designed as a CAR recognizing the antigen 1 confirmed to be expressed based on the result of FIG. 1D, the corresponding CAR-T attacks only a specific clone in tumor cells and prevents other clones in which the corresponding antigen is not expressed from being removed. The same is true of CART-NK.

In the end, it is difficult to design a CAR with high coverage when the expression of an antigen is not verified based on a single cell as described above, but decided to a population level according to a conventional method. To design a CAR with wide coverage and high specificity, effective target antigen discovery is important. Hereinafter, a new methodology for target antigen discovery will be described.

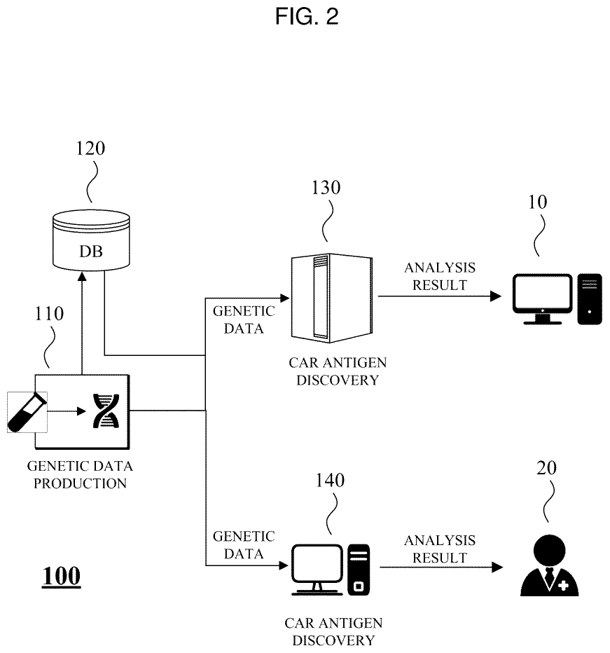

FIG. 2 illustrates an example of a target antigen discovery system 100 for a CAR. Analysis apparatuses 130 and 140 are apparatuses for discovering a target antigen for a CAR. In FIG. 2, the analysis apparatuses are illustrated in the form of a server 130 and a computer terminal 140. The server 130 may provide a service of discovering a target antigen on a network. The computer terminal 140 may be connected to a network or discover a target antigen as a discrete apparatus. Meanwhile, the analysis apparatuses 130 and 140 may be realized in various forms.

A gene analysis apparatus 110 produces genetic data of a sample by analyzing the sample. A gene database (DB) 120 may store and manage the produced genetic data. The genetic data may include a genetic sequence and gene expression information of the sample. The genetic data includes the expression information on antigen genes on the surface of a tumor cell. The genetic data includes information on the expression levels of antigen genes. On the other hand, each of the plurality of genes corresponds to one antigen.

The gene DB 120 may be an open DB possessing the results of experiments conducted by multiple researchers. Meanwhile, the gene DB 120 may possess genetic data of the sample of a specific patient. In this case, the analysis apparatuses 130 and 140 may discover a patient-specific antigen.

The analysis apparatuses 130 and 140 analyze genetic data to discover a target antigen for a CAR. A process of discovering a target antigen by the analysis apparatuses 130 and 140 will be described.

Users 10 and 20 may confirm the results of discovering a target antigen for a specific tumor or specific patient. The user 10 may log in to a server 130 through a user terminal (PC, smartphone or the like) to confirm the analysis result accomplished by the server 130. The user 20 may confirm a target antigen discovery result through the computer terminal 140 used by the user 20.

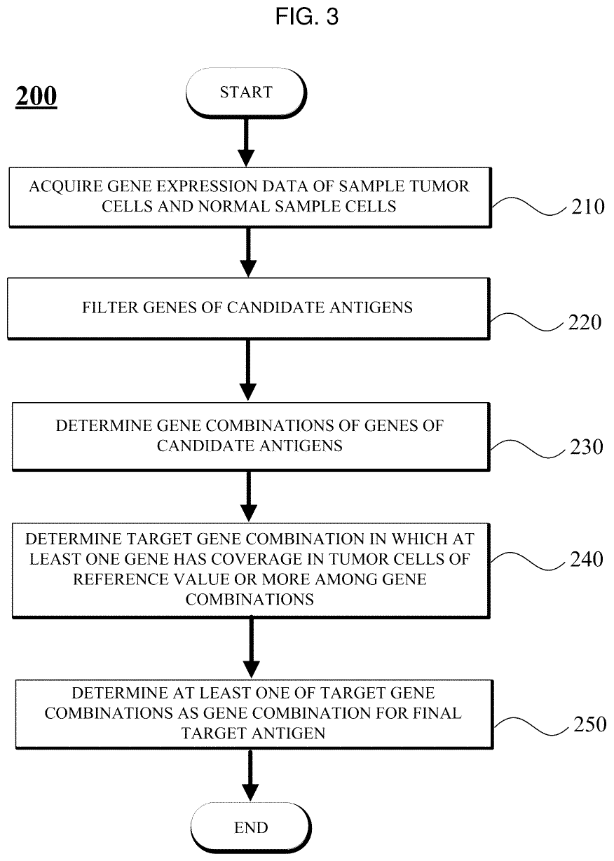

FIG. 3 illustrates one example of a flowchart illustrating a process (200) of discovering a target antigen for a CAR.

The analysis apparatus acquires gene expression information of sample tumor cells (210). Here, the sample may be samples of a specific type of tumor (e.g., lung cancer) collected from multiple persons. Alternatively, the sample may be cells taken from a specific patient. The analysis apparatus may also acquire gene expression information of normal sample cells (210). Information on normal cells may be used for specificity evaluation. Here, tumor cells and normal cells may be cells acquired from various cell populations, rather than a single clone.

The genetic data of tumor cells and/or normal cells collected by the analysis apparatus is referred to as source data. The source data includes gene expression information in a discrete cell unit through single cell analysis. Tumor cells included in the source data may also be referred to as a tumor cell group. In addition, the normal cells included in the source data may also be referred to as a normal cell group.

The analysis apparatus may filter effective data from the source data (220). The analysis apparatus may select genes of candidate antigens from the source data (220). The candidate antigens correspond to antigens that are a population for discovering a target antigen. A gene of a candidate antigen is a gene directly or indirectly involved in the expression of a corresponding antigen. The gene(s) involved in expression of a candidate antigen may be referred to as candidate gene(s). In addition, a set of genes involved in expression of each candidate antigen may be referred to as a candidate gene group.

For the selection of a candidate gene, a variety of criteria may be used. (i) An analysis apparatus may select a gene expressed at a certain rate or more as a candidate gene in cells included in a tumor cell group based on the tumor cell group. For example, the analysis apparatus may select a gene for an antigen expressed at 1% or more in the tumor cell group as a candidate gene. (ii) The analysis apparatus may select a gene that has a certain difference in expression pattern in tumor cells and normal cells as a candidate gene. For example, the analysis apparatus may analyze a differential expression gene (DEG) in tumor cells, compared to normal cells, as a candidate gene. The analysis apparatus may select a gene whose expression level in tumor cells is the same as or higher than the reference value compared to that of normal cells. (iii) Further, the analysis apparatus may select a candidate gene by combining the criterion (i) and the criterion (ii). For example, the analysis apparatus may select a gene differentially expressed greater than a certain standard in tumor cells among the genes for an antigen expressed 1% or greater in a tumor cell group as a candidate gene.

The analysis apparatus determines all combinations that can consist of candidate genes based on the filtered genes of the candidate antigens (candidate genes) (230). The analysis apparatus may determine all available combinations mathematically according to the number of cases. Alternatively, the analysis apparatus may determine a possible gene combination for genes substantially expressed simultaneously considering the expression relationship of genes.

The analysis apparatus confirms whether at least one gene included in each gene combination is expressed greater than the reference rate in tumor cells. The analysis apparatus determines a target gene combination based on a rate (coverage) of cells in which at least one gene included in each combination is expressed among all tumor cells (240). For example, when tumor cells in which at least one gene included in the combination is expressed account for 60% or more of all of the tumor cells, the analysis apparatus may determine the corresponding combination as a target gene combination. The target gene combination includes a gene involved in the expression of a target gene recognized by a CAR. At least one gene included in the gene combination is involved in the expression of one target antigen.

The analysis apparatus determines the final gene combination for a target antigen among the target gene combinations (250). (i) The analysis apparatus may determine at least one of the top-rated gene combinations in terms of coverage from the target gene combinations as the final gene combination. (ii) The analysis apparatus may determine at least one of the top-rated multiple gene combinations in terms of coverage from the target gene combinations, in which coverage in normal cells is less than the reference value, as the final gene combination.

In FIG. 3, the analysis apparatus discovers a target antigen based on at least one antigen expressed in tumor cells. That is, when at least one gene in a gene combination has coverage the same as or greater than the reference value in tumor cells, the analysis apparatus may determine that the corresponding gene is involved in an effective candidate antigen. In other words, when at least one antigen is expressed at a certain rate or more in tumor cells, the analysis apparatus determines the corresponding antigen as a target antigen.

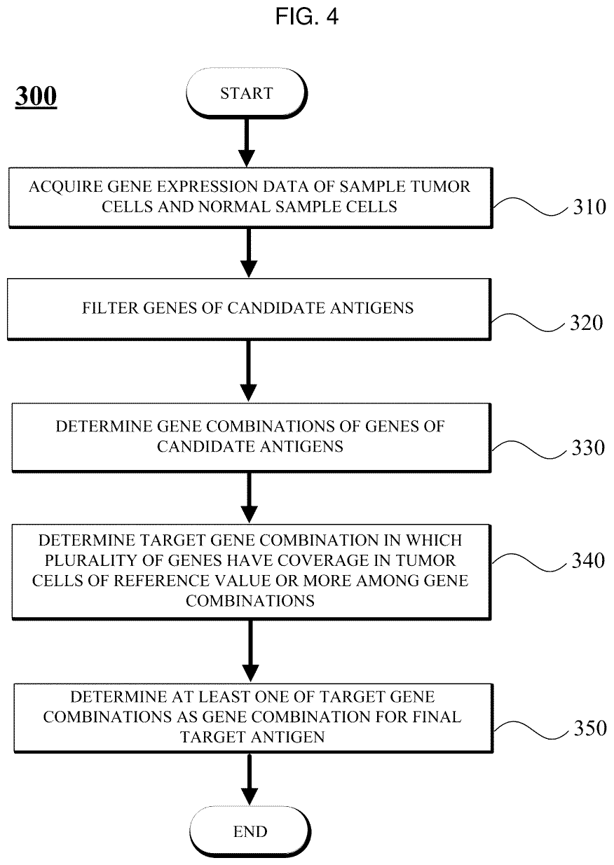

FIG. 4 illustrate another example of a flowchart illustrating a process (300) of discovering a target antigen for a CAR.

An analysis apparatus acquires gene expression information of sample tumor cells (310). Here, the sample may be samples of a specific type of tumor (e.g., lung cancer) collected from multiple persons. Alternatively, the sample may be cells taken from a specific patient. The analysis apparatus may also acquire gene expression information of normal sample cells (310). Information on normal cells may be used for specificity evaluation. Here, tumor cells and normal cells may be cells acquired from various cell populations, rather than a single clone.

The analysis apparatus may filter effective data from the source data (320). The analysis apparatus may select genes of candidate antigens (candidate genes) from the source data (320).

For the candidate gene selection, a variety of criteria may be used. (i) An analysis apparatus may select a gene expressed at a certain rate or more as a candidate gene in cells included in a tumor cell group based on the tumor cell group. For example, the analysis apparatus may select a gene for an antigen expressed at 1% or more in the tumor cell group as a candidate gene. (ii) The analysis apparatus may select a gene that has a certain difference in expression pattern in tumor cells and normal cells as a candidate gene. For example, the analysis apparatus may analyze a differential expression gene (DEG) in tumor cells, compared to normal cells, as a candidate gene. The analysis apparatus may select a gene whose expression level in tumor cells is the same as or higher than the reference value compared to that of normal cells. (iii) Further, the analysis apparatus may select a candidate gene by combining the criterion (i) and the criterion (ii). For example, the analysis apparatus may select a gene differentially expressed greater than a certain standard in tumor cells among the genes for an antigen expressed 1% or greater in a tumor cell group as a candidate gene.

The analysis apparatus determines all combinations that can consist of candidate genes based on the filtered genes (candidate genes) of the candidate antigens (candidate genes) (330). The analysis apparatus may determine all available combinations mathematically according to the number of cases. Alternatively, the analysis apparatus may determine a possible gene combination for genes expressed substantially simultaneously considering the expression relationship of genes.

The analysis apparatus confirms whether at least one gene included in each gene combination is expressed greater than the reference rate in tumor cells. The analysis apparatus determines a target gene combination based on coverage of cells in which at least one gene included in each combination is expressed among all tumor cells (340). For example, when tumor cells in which at least one gene included in the combination is expressed account for 60% or more of all of the tumor cells, the analysis apparatus may determine the corresponding combination as a target gene combination. The target gene combination includes a gene involved in the expression of a target gene recognized by CAR. At least one gene included in the gene combination is involved in the expression of one target antigen. A plurality of genes may be involved in expression of one antigen. However, here, it is assumed that each of the plurality of genes may express a different antigen.

The analysis apparatus determines the final gene combination for a target antigen among the target gene combinations (350). (i) The analysis apparatus may determine at least one of the top-rated gene combinations in terms of coverage from the target gene combinations as the final gene combination. (ii) The analysis apparatus may determine at least one of the top-rated multiple gene combinations in terms of coverage from the target gene combinations, in which coverage in normal cells is less than the reference value, as the final gene combination.

In FIG. 4, the analysis apparatus discovers a target antigen based on at least one antigen expressed in tumor cells. That is, when at least one gene in a gene combination has coverage the same as or greater than the reference value in tumor cells, the analysis apparatus may determine that the corresponding gene is involved in an effective candidate antigen.

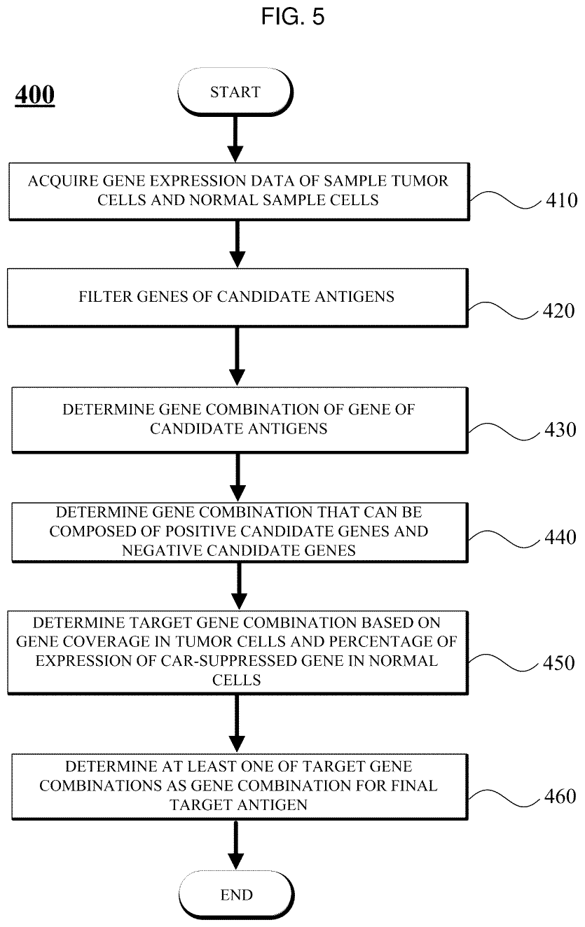

FIG. 5 illustrates still another example of a flowchart illustrating a process (400) of discovering a target antigen for a CAR.

An analysis apparatus acquires gene expression information of sample tumor cells (410). Here, the sample may be samples of a specific type of tumor (e.g., lung cancer) collected from multiple persons. Alternatively, the sample may be cells taken from a specific patient. The analysis apparatus may also acquire gene expression information of normal sample cells (410). Information on normal cells may be used for specificity evaluation. Here, tumor cells and normal cells may be cells acquired from various cell populations, rather than a single clone.

The analysis apparatus may filter effective data from the source data (420). The analysis apparatus may select genes of candidate antigens from the source data (420).

For the candidate gene selection, a variety of criteria may be used. (i) An analysis apparatus may select a gene expressed at a certain rate or more as a candidate gene in cells included in a tumor cell group based on the tumor cell group. For example, the analysis apparatus may select a gene for an antigen expressed at 1% or more in the tumor cell group as a candidate gene. (ii) The analysis apparatus may select a gene that has a certain difference in expression pattern in tumor cells and normal cells as a candidate gene. For example, the analysis apparatus may analyze a differential expression gene (DEG) in tumor cells, compared to normal cells, as a candidate gene. The analysis apparatus may select a gene whose expression level in tumor cells is higher than the reference value compared to that of normal cells. (iii) Further, the analysis apparatus may select a candidate gene by combining the criterion (i) and the criterion (ii). For example, the analysis apparatus may select a gene differentially expressed greater than a certain standard in tumor cells among the genes for an antigen expressed 1% or greater in a tumor cell group as a candidate gene.

The analysis apparatus determines positive candidate genes and negative candidate genes in genes of the candidate antigens (430). The positive candidate gene is a gene associated with an antigen expressed tumor cell-specifically or highly in tumor cells. (i) The analysis apparatus may select a gene having coverage the same as or greater than the reference value in tumor cells among the genes of the candidate antigens as a positive candidate gene. (ii) Alternatively, the analysis apparatus may select a gene expressed in tumor cells whose number is the same as or higher than the reference value among the sample tumor cells as a positive candidate gene.

When determining the positive candidate gene, the analysis apparatus determines at least one negative candidate gene with respect to the corresponding positive candidate gene. To this end, the analysis apparatus has to possess information on the correlation in expression of genes. That is, the information on gene correlation is information on whether the expression of a specific gene promotes or inhibits the expression of at least one gene. The analysis apparatus may use transcriptome information to identify the information on the correlation between genes. The negative candidate gene is a gene whose expression pattern has a negative relationship with the positive candidate gene. That is, the negative candidate gene is a gene whose expression level is reduced when the positive candidate gene is highly expressed. The analysis apparatus may determine a negative candidate gene whose expression pattern has a negative relationship with the determined positive candidate gene. The analysis apparatus may also determine a plurality of negative candidate genes with respect to one positive candidate gene.

The analysis apparatus determines all gene combinations that can consist of positive candidate genes and negative candidate genes (440). The analysis apparatus may determine all available combinations mathematically according to the number of cases. Alternatively, the analysis apparatus may determine a possible gene combination for genes expressed substantially simultaneously considering the expression relationship of genes.

The analysis apparatus confirms whether at least one gene included in each gene combination is expressed greater than the reference rate in tumor cells. The analysis apparatus determines a target gene combination based on a rate (coverage) of cells in which at least one gene included in each combination is expressed among all tumor cells (450). Further, the analysis apparatus may determine a target gene combination based on the expression rate of a gene suppressing a CAR in normal cells (450).

Several examples for the analysis apparatus determining a target gene combination will be described. (i) When tumor cells in which at least one positive candidate gene included in a combination is expressed are 60% (merely an example of the reference value) or more of all tumor cells, an analysis apparatus may determine the corresponding combination as a target gene combination. (ii) When tumor cells in which at least one negative candidate gene included in a combination is expressed are less than 10% of all tumor cells, the analysis apparatus may determine the corresponding combination as a target gene combination. (iii) When tumor cells in which at least one positive candidate gene included in a combination is expressed are 60% or more of all tumor cells, the analysis apparatus may determine the corresponding combination as a target gene combination. Here, since, during preparation, CAR-T or CAR-NK has to be designed to inhibit an immune response when a CAR binds to an antigen of a negative gene, the negative gene may be referred to as an immunosuppressive gene and serve to minimize damage to normal cells. (iv) When tumor cells in which at least one positive candidate gene included in a combination is expressed are 60% or more of all tumor cells, the analysis apparatus may determine the corresponding combination as a primary target gene combination. Afterward, the analysis apparatus may determine the primary target gene combination as a population and then as the final target gene combination. For example, among the primary target gene combinations, the analysis apparatus may determine a combination in which normal cells in which an immunosuppressive gene is expressed account for 60% or more of the normal cells in which at least one positive candidate gene included in each combination is expressed as a secondary target gene combination. The secondary target gene combination may be the final target gene combination.

The analysis apparatus finally determines a gene combination for a target antigen among the target gene combinations (460). (i) The analysis apparatus may determine at least one of the top-rated gene combinations in terms of coverage from the target gene combinations as the final gene combination. (ii) The analysis apparatus may determine at least one of the top-rated multiple gene combinations in terms of coverage from the target gene combinations, in which coverage in normal cells is less than the reference value, as the final gene combination.

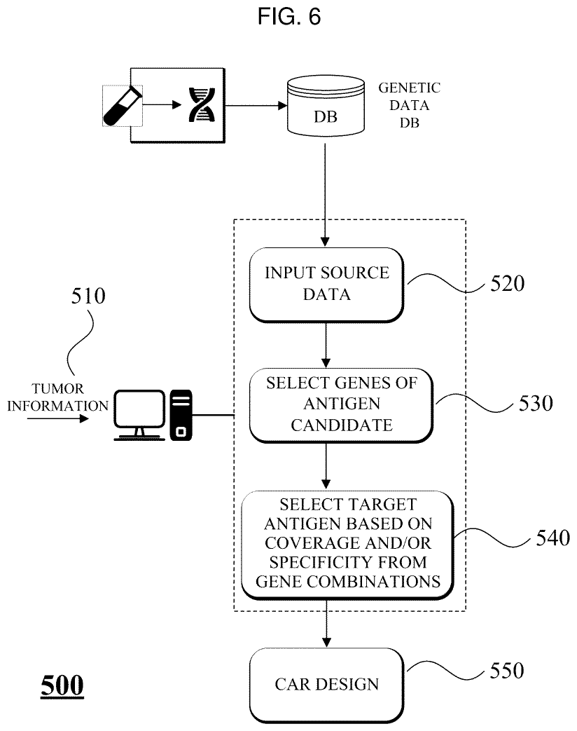

FIG. 6 illustrates an example of a process (500) of discovering a target antigen by an analysis apparatus. The gene DB stores gene expression information of tumor cells. The gene DB may store gene expression information of normal cells. In addition, the gene DB may store information on the correlation in expression of genes.

The analysis apparatus may receive desired tumor information (510). The tumor information may include a tumor type, clinical information, and patient information. (i) The tumor information may define a specific tumor with specific clinical conditions. The clinical information may include a race, sex, and age of a sample, the stage of tumor progression (early, intermediate, late, etc.), and an anatomical area from which the sample is extracted. In this case, the tumor information will define a specific tumor with specific clinical conditions. (ii) In addition, the tumor information may define a specific tumor in a specific patient.

The analysis apparatus may input or receive source data corresponding to the tumor information received from the gene DB (520). The source data may consist of RNA expression data of tumor cells corresponding to tumor information (at least one criterion of a race, a sex, and tissue) and normal cells manipulated as above.

The analysis apparatus may select genes of antigen candidates from the source data (530). Gene filtering of an antigen candidate is as described above. Meanwhile, the process of selecting candidate genes may be omitted.

The analysis apparatus organizes a possible gene combination based on the selected genes, or genes received from the gene DB. Afterward, the analysis apparatus may select a target gene combination based on the coverage of a gene included in the corresponding combination from all possible gene combinations (540). Alternatively, the analysis apparatus may select a target gene combination based on the specificity of a gene included in the corresponding combination from all possible gene combinations (540). The processes of determining a target gene combination have been described above with reference to FIGS. 3 to 5. The analysis apparatus may select a target gene combination itself, or a more effective specific gene combination among the target gene combinations, as the final gene combination or gene for a target antigen (540).

Afterward, the analysis apparatus may deliver the final target antigen information for a CAR to another object. Alternatively, the analysis apparatus may design a recognition site for a CAR recognizing the final target antigen (550).

The in silico experimental processes and results for a process of discovering a target antigen based on actual genetic data will be described below.

Source data was prepared by collecting the following data. Proteome analysis data for 41 cell types obtained by mass spectrometry, transcriptome data from 610 tumor cell lines, 2,769 tumor cell membrane protein genes extracted from the human surfaceome resource (Bausch-Fluck et al. PNAS, 2018) built through machine learning, proteome data and transcriptome data for 375 tumor cell lines (Nusinow et al. Cell, 2020), and 1,421 cell membrane protein genes extracted from a gene ontology database are used in the experimental process.

Source data included 3,687 non-redundant genes and expression information on the corresponding genes.

Lung cancer and colorectal cancer are used as research target. Based on lung cancer LUAD and LUSC, 498 genes differentially expressed in tumor cells were selected from genes present in tumor cells and normal cells by the researchers. In addition, based on colorectal cancer COAD and READ, 315 genes differentially expressed in tumor cells were selected from genes present in tumor cells and normal cells by the researchers.

Single cell transcriptome data of lung cancer included 58 samples obtained from 44 patients. Transcriptome data of colorectal cancer included 33 samples obtained from 23 patients. Tumor epithelial cells were isolated through single cell transcriptome analysis technology, and other immune cells were considered normal cells. Lung cancer cells consisted of 165,511 tumor cells and 42,995 normal cells, and colorectal cancer cells consisted of 47,285 tumor cells and 16,404 normal cells.

Hereinafter, the process expressed as being conducted by the “researchers” is a process performed by all researchers using a computer device, such as an “analysis apparatus.”

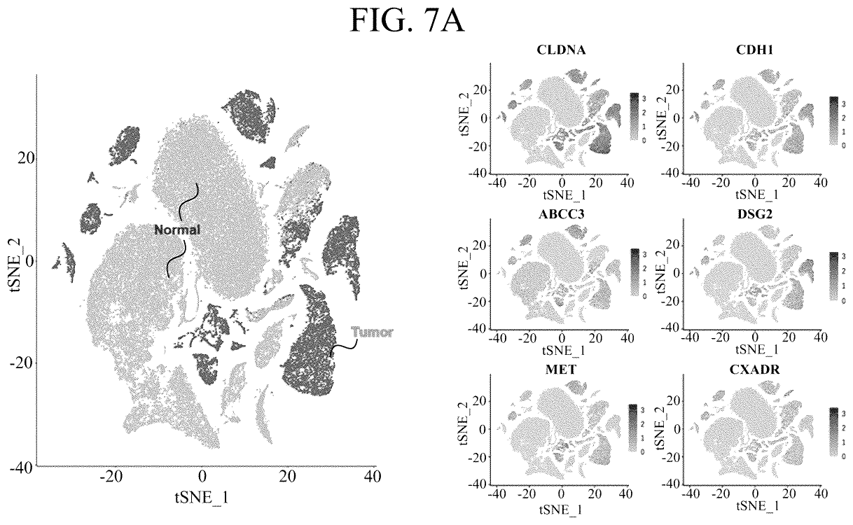

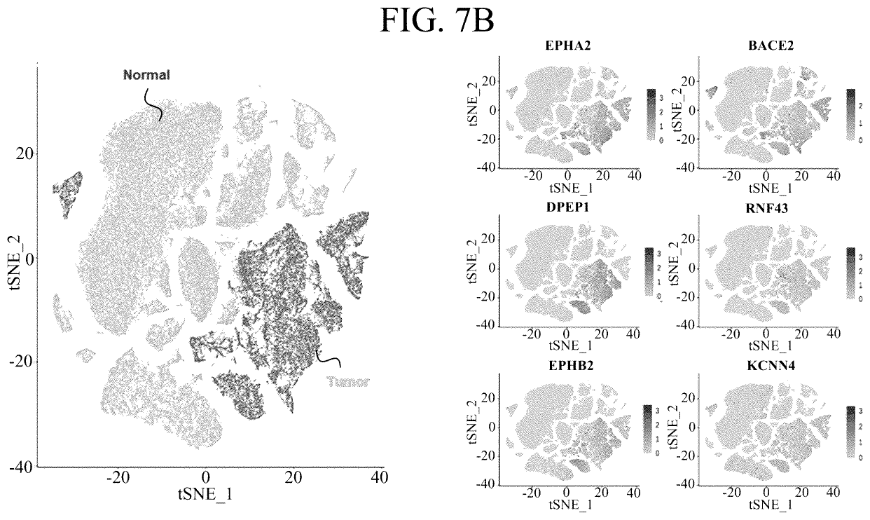

Genes of candidate antigens were first filtered from the source data by the researchers. Genes exhibiting at least 1% or more gene expression (cell coverage) in tumor cells were selected through single cell analysis by the researchers. In addition, genes whose expression level itself showed a difference of the reference value or greater were selected through DEG analysis between normal cells and tumor cells by the researchers. Finally, 163 genes of lung cancer and 110 genes of colorectal cancer were secured by the researchers. FIG. 7 is an experimental result showing the gene expression patterns of tumor cells and normal cells in a colorectal cancer sample and a lung cancer sample. FIG. 7 illustrates the t-stochastic neighbor embedding (SNE) results, expressing the gene expression patterns through dimensionality reduction. FIG. 7A is the expression pattern of 163 lung cancer genes. The left panel of FIG. 7A shows a difference in expression between tumor cells and normal cells based on entire genes, and the right panel of FIG. 7A shows an expression difference based on a specific gene. FIG. 7B is the expression patterns of 110 genes. The left panel of FIG. 7B shows a difference in expression between tumor cells and normal cells based on entire genes, and the right panel of FIG. 7B shows an expression difference based on a specific gene.

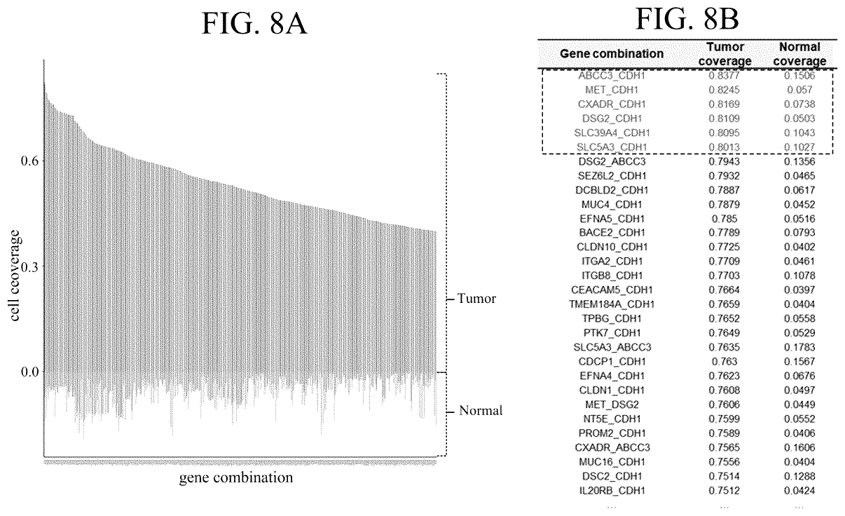

From 163 lung cancer genes, 70 genes having coverage in tumor cells of 10% or more, except ‘CLDN4 (93.5%)’ genes having coverage in tumor cells of 90% or more, were selected by the researchers. 2,415 gene combinations were determined using 70 genes by the researchers. Whether the gene expression was in a single cell unit, in which at least one of the genes included in each gene combination was expressed, was converted into binary information by the researchers. 253 combinations having coverage in tumor cells of 60% or more were selected by the researchers. FIGS. 8A and 8B illustrate an experimental result for a gene combination in which one or more genes are expressed in a lung cancer sample. FIG. 8A shows the expression rates of 253 gene combinations in tumor cells and normal cells. FIG. 8B shows tumor combinations having relatively high coverage in tumor cells, and the top-rated combinations having the coverage of 80% or more are indicated by dotted boxes. In this case, the analysis apparatus may select the gene combination having the highest coverage in tumor cells among the top-rated combinations as a final antigen candidate. In addition, the analysis apparatus may select the gene combination having low coverage in normal cells among the gene combinations having high coverage in tumor cells as a final antigen candidate. In summary, the analysis apparatus may select the final antigen candidate based on at least one of the coverage in tumor cells and the coverage in normal cells.

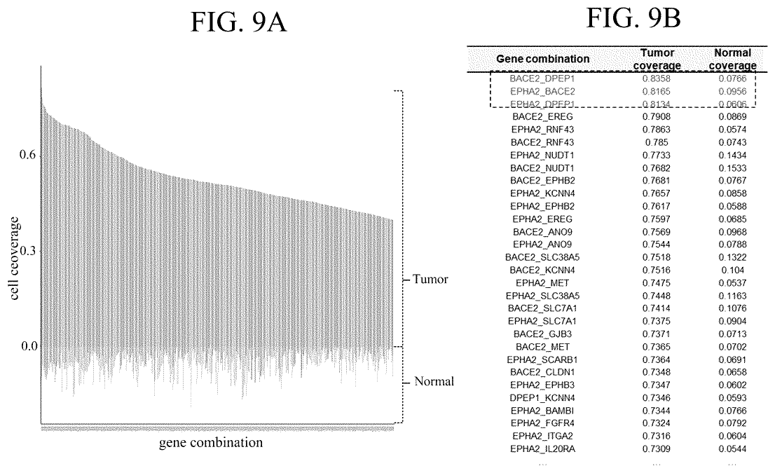

Genes having coverage in tumor cells of 10% or more were selected from 110 colorectal cancer genes by the researchers. 2,278 gene combinations were determined using the selected genes by the researchers. Whether the gene expression was in a single cell unit, in which at least one of the genes included in each gene combination was expressed, was converted into binary information by the researchers. 248 combinations having coverage in tumor cells of 60% or more were selected by the researchers. FIGS. 9A and 9B illustrate an experimental result for a gene combination in which one or more genes are expressed in a colorectal cancer sample. FIG. 9A shows the expression rates of 248 gene combinations in tumor cells and normal cells. FIG. 9B shows the gene combinations having relatively high coverage in tumor cells, and the top-rated combinations having the coverage of 80% or more are indicated by dotted boxes. In this case, the analysis apparatus may select the gene combination having the highest coverage in tumor cells among the top-rated combinations as a final antigen candidate. In addition, the analysis apparatus may select the gene combination having low coverage in normal cells among the gene combinations having high coverage in tumor cells as a final antigen candidate. In summary, the analysis apparatus may select the final antigen candidate based on at least one of the coverage in tumor cells and the coverage in normal cells.

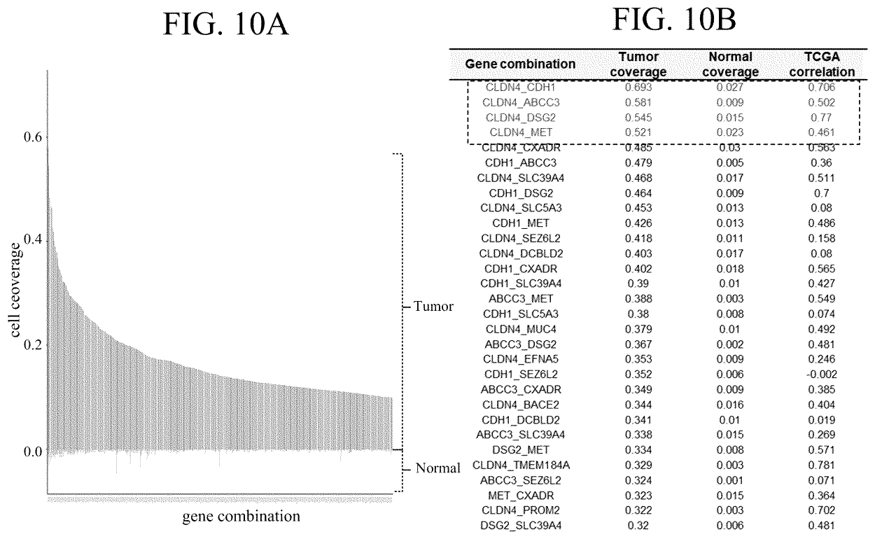

Genes having coverage in tumor cells of 10% or more were selected from 163 lung cancer genes by the researchers. 2,415 gene combinations were determined using the selected genes by the researchers. Whether the gene expression was in a single cell unit, in which two or more genes included in each gene combination were simultaneously expressed, was converted into binary information by the researchers. 589 combinations having coverage in tumor cells of 10% or more were selected by the researchers. FIGS. 10A and 10B illustrate an experimental result for a gene combination in which two or more genes are expressed in a lung cancer sample. FIG. 10A shows the expression rates of 589 gene combinations in tumor cells and normal cells. FIG. 10B shows tumor combinations having relatively high coverage in tumor cells, and the top-rated combinations having the coverage of 50% or more are indicated by dotted boxes. In this case, the analysis apparatus may select the gene combination having the highest coverage in tumor cells among the top-rated combinations as a final antigen candidate. In addition, the analysis apparatus may select the gene combination having low coverage in normal cells among the gene combinations having high coverage in tumor cells as a final antigen candidate. In addition, the analysis apparatus may select the gene combination having a high correlation in expression between two or more co-expressed genes. In summary, the analysis apparatus may select the final antigen candidate based on at least one of coverage in tumor cells, coverage in normal cells, and the correlation in expression of genes.

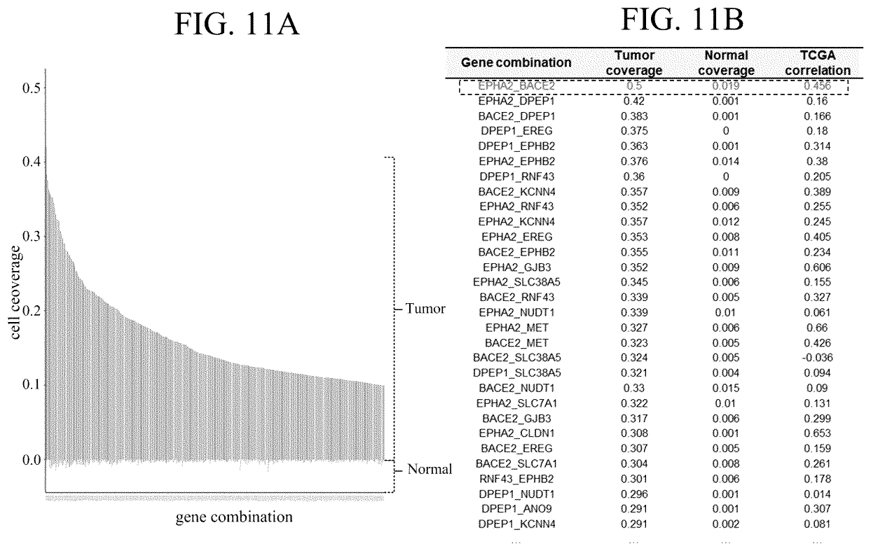

Genes having coverage in tumor cells of 10% or more were selected from 110 colorectal cancer genes by the researchers. 2,278 gene combinations were determined using the selected genes by the researchers. Whether the gene expression was in a single cell unit, in which at least one of the genes included in each gene combination was expressed, was converted into binary information by the researchers. 537 combinations having coverage in tumor cells of 60% or more were selected by the researchers. FIGS. 11A and 11B illustrate an experimental result for a gene combination in which two or more genes are expressed in a colorectal cancer sample. FIG. 11A shows the expression rates of 537 gene combinations in tumor cells and normal cells. FIG. 11B shows the gene combinations having a relatively high coverage in tumor cells, and the top-rated combinations having the coverage of 50% or more are indicated by dotted boxes. In this case, the analysis apparatus may select the gene combination having the highest coverage in tumor cells among the top-rated combinations as a final antigen candidate. In addition, the analysis apparatus may select the gene combination having low coverage in normal cells among the gene combinations having high coverage in tumor cells as a final antigen candidate. In addition, the analysis apparatus may select the gene combination having a high correlation in expression between two or more co-expressed genes. In summary, the analysis apparatus may select the final antigen candidate based on at least one of coverage in tumor cells, coverage in normal cells, and the correlation in expression of genes.

All co-expression indexes between gene combinations in tumors were calculated using 10,459 transcriptome sequence data obtained from a TCGA DB by the researchers. Possible combinations were drawn from genes (negative candidate genes) having a negative correlation with six main candidate genes (positive candidate genes), selected from lung cancer and colorectal cancer cells by the researchers. A protection rate was used to evaluate the specificity of a CAR by the researchers. The protection rate is a rate of expressing a tumor cell-specific gene (positive gene), together with an immunosuppressive gene (negative gene) having a suppressive action on a CAR, in normal cells. Here, during preparation, CAR-T or CAR-NK has to be designed to inhibit an immune response when a CAR binds to an antigen and may serve to minimize damage to normal cells. For the suppressive gene, the researchers' own experimental results or results from a previous study may be used according to the characteristics of a CAR.

Main positive candidate genes were selected from lung cancer genes by the researchers. As the main positive candidate genes, six genes having high coverage were selected based on coverage in tumor cells. In addition, the researchers selected negative candidate genes whose expression pattern had a negative relationship with the positive candidate genes. One or more negative candidate genes may be selected for one positive candidate gene. From all gene combinations that can consist of the selected positive candidate genes and negative candidate genes, 462 combinations having coverage in tumor cells of 60% or more were selected by the researchers.

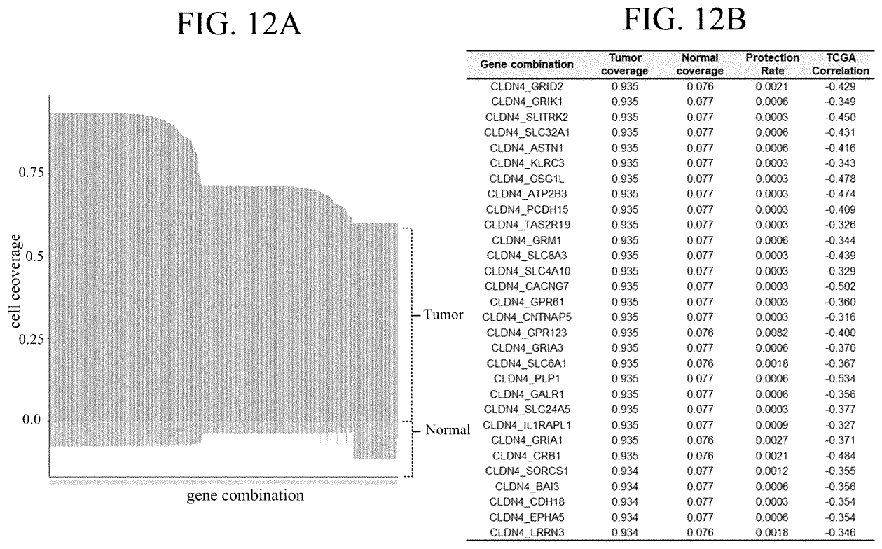

FIGS. 12A and 12B illustrate an experimental result for a tumor cell-specific gene combination in a lung cancer sample. FIG. 12A shows the expression rates of 462 gene combinations in tumor cells and normal cells. In FIG. 12B, gene combinations having relatively high coverage in tumor cells are indicated. FIG. 12B shows values of items, such as coverage in normal cells (tumor coverage), coverage in normal cells (normal coverage), a protection rate, and the correlation in expression of genes included in a gene combination (TCGA correlation). In this case, the analysis apparatus may select a gene combination having tumor coverage higher than a certain value as a final antigen candidate. In addition, the analysis apparatus may select a gene combination having normal coverage lower than a certain level among gene combinations having high tumor coverage as a final antigen candidate. In addition, the analysis apparatus may select a gene combination having a protection rate higher than a certain level among gene combinations having high tumor coverage as a final antigen candidate. In addition, the analysis apparatus may select a gene combination having a high correlation in expression between a positive candidate gene and a negative candidate gene as a final antigen candidate. In summary, the analysis apparatus may select a final antigen candidate based on a combination of tumor coverage, normal coverage, a protection rate, and the correlation in expression of genes.

Main positive candidate genes were selected from colorectal cancer genes by the researchers. As the main positive candidate genes, six genes having high coverage were selected based on tumor coverage. In addition, negative candidate genes whose expression pattern had a negative relationship with each positive candidate gene were selected by the researchers. One or more negative candidate genes may be selected for one positive candidate gene. From all gene combinations which can consist of the selected positive candidate genes and negative candidate genes, 449 combinations having tumor coverage of 40% or more were selected by the researchers.

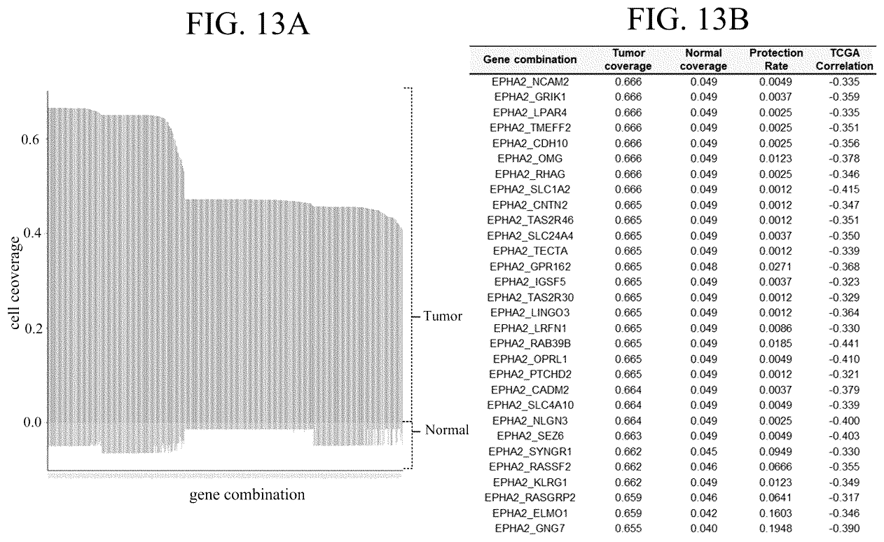

FIGS. 13A and 13B illustrate an experimental result for a tumor cell-specific gene combination in a colorectal cancer sample. FIG. 13A shows the expression rates of 462 gene combinations in tumor cells and normal cells. In FIG. 13B, gene combinations having relatively high coverage in tumor cells are indicated. FIG. 13B shows values of items, such as coverage in tumor cells (tumor coverage), coverage in normal cells (normal coverage), a protection rate, and the correlation in expression of genes included in a gene combination (TCGA correlation). In this case, the analysis apparatus may select a gene combination having tumor coverage higher than a certain value as a final antigen candidate. In addition, the analysis apparatus may select a gene combination having normal coverage lower than a certain value among the gene combinations having high tumor coverage as a final antigen candidate. In addition, the analysis apparatus may select a gene combination having a protection rate higher than a certain value among the gene combinations having high tumor coverage as a final antigen candidate. In addition, the analysis apparatus may select a gene combination having a high correlation in expression between a positive candidate gene and a negative candidate gene as a final antigen candidate. In summary, the analysis apparatus may select a final antigen candidate based on a combination of tumor coverage, normal coverage, a protection rate and the correlation in expression of genes.

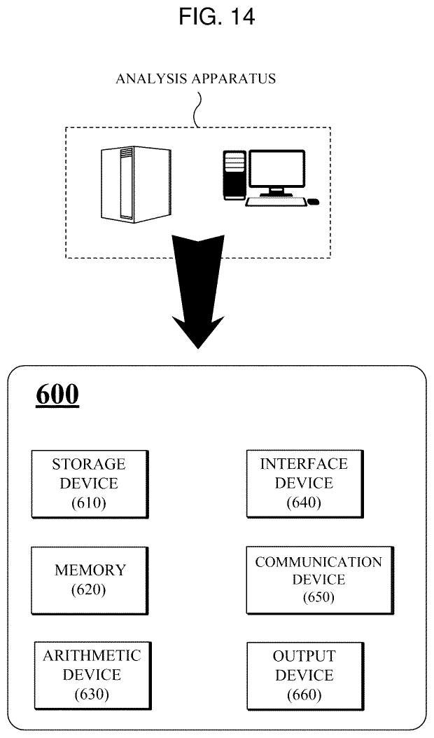

FIG. 14 is an example of an analysis apparatus for discovering an antigen for a CAR.

An analysis apparatus 600 is an apparatus that corresponds to the analysis apparatus 130 or 140 in FIG. 1. The analysis apparatus 600 discovers a target antigen recognized by a CAR.

The analysis apparatus 600 may be realized in various physical forms. For example, the analysis apparatus 600 may have a form such as a computer device like PC, a network server, or a data processing-exclusive chipset. Computer devices may include a mobile device such as a smart device.

The analysis apparatus 600 may include a storage device 610, a memory 620, an arithmetic device 630, an interface device 640, a communication device 650, and an output device 660.

The storage device 610 stores a program for determining an antigen recognized by a CAR using expression information of genes in tumor cells. The corresponding program may determine an antigen recognized by a CAR using gene expression information in not only tumor cells but also normal cells. Further, the storage device 610 may store programs or source codes necessary for data processing. The storage device 610 may store input genetic data and/or tumor information necessary for analysis.

The memory 620 may store data and information, which are produced in the process of analyzing the genetic data by the analysis apparatus 600.

The interface device 640 is an apparatus that receives certain commands and data from the outside. The interface device 640 may receive genetic data from a physically connected input device or external storage device. The interface device 640 may receive expression data of genes in tumor cells and normal cells in a single cell unit. The interface device 640 may also receive analysis criteria, tumor information, etc.

The communication device 650 is a component that receives and transmits certain information via a wired or wireless network. The communication device 650 may receive genetic data from an external object. The communication device 650 may receive expression data of genes in tumor cells and normal cells in a single cell unit. The communication device 650 may transmit target antigen-related information, which is an analysis result, to an external object.

The communication device 650 or the interface device 640 is an apparatus that receives certain data or commands from the outside. The communication device 650 or the interface device 640 may be referred to as an input device.

The output device 660 is an apparatus that outputs certain data. The output device 660 may output an interface, an analysis result, etc., necessary for data processing.

The arithmetic device 630 may discover a target antigen by analyzing genetic data using the program stored in the storage device 610.

The arithmetic device 630 may select genes of candidate antigens (candidate genes) from source data. The criteria and procedure for candidate gene selection are as follows.

The arithmetic device 630 may determine all initial gene combinations which can consist of a plurality of genes among the genes of the candidate antigens. The arithmetic device 630 may determine a target gene combination associated with a target antigen from the initial gene combinations. In addition, the arithmetic device 630 may select an optimal final gene combination or gene when there are a plurality of gene combinations in the target gene combinations.

The arithmetic device 630 may select a combination in which a rate (coverage) of cells in which at least one gene included in an initial gene combination is expressed among tumor cells is the same as or greater than the reference value as a target gene combination.

The arithmetic device 630 may select a combination in which a rate (coverage) of cells in which a plurality of genes included in an initial gene combination is expressed among tumor cells is the same as or greater than the reference value as a target gene combination.

The arithmetic device 630 may determine a positive candidate gene from genes of candidate antigens based on the percentage of cells in which a gene is expressed in tumor cells or the number of cells in which a gene is expressed in tumor cells. The arithmetic device 630 may determine negative candidate genes whose expression pattern has a reverse relationship with the positive candidate genes.

The arithmetic device 630 may determine all gene combinations that can consist of positive candidate genes and negative candidate genes as an initial gene combination.

The arithmetic device 630 may determine a combination in which the percentage of cells in which at least one positive candidate gene of the positive candidate genes included in a specific combination is expressed among tumor cells is the same as or greater than the reference value, and

the percentage of normal cells in which a suppressive gene suppressing CAR recognition or linkage with a positive candidate gene-involved antigen among normal cells in which at least one positive candidate gene is expressed is the same as or greater than the reference value as a target gene combination.

The arithmetic device 630 may be an apparatus for processing data and certain calculations, such as a processor, AP, or a program-embedded chip.

Further, while performing subsequent research, the researchers developed an algorithm for automating a target antigen discovery process for a CAR. The corresponding algorithm uses a machine learning model. Hereinafter, a process of building the corresponding algorithm or machine learning model by the researchers will be described. In the following description, genes include cell surface genes as tumor-associated antigen genes.

First, a large-scale transcriptome database for single cells was established by the researchers. An independent big database (Single cell Tumor-Normal Meta Atlas) was established by integrating single cell RNA-seq data for 17 carcinomas and 12 normal tissues by the researchers.

The 17 carcinomas included anaplastic thyroid cancer (ATC), breast cancer (BRCA), colorectal cancer (CRC), gastric cancer (GC), gastrointestinal neuroendocrine tumor (GI-NET), gastrointestinal stromal tumor (GIST), glioblastoma (GBM), head and neck cancer (HN), liver hepatocellular carcinoma (LIHC), intrahepatic cholangiocarcinoma (IH-CCA), non-small-cell lung carcinoma (NSCLC), non-Hodgkin's lymphoma (NHL), ovarian cancer (OV), pancreatic adenocarcinoma (PAAD), prostate cancer (PC), sarcoma (SARC), and uveal melanoma (UVM).

The 12 normal tissues include breast, large intestine, fallopian tube and ovary, heart, ileum, kidney, liver, lung, pancreas, prostate, rectum, and eye. The subdivided tissues include adipose tissue, skeletal muscle, placenta, bronchus, skin, esophagus, lymph node, testis, small intestine, stomach, peripheral blood mononuclear cell population, spleen, and bone marrow.

The cancer cell atlas (CCA) created by the researchers using cancer tissue data consisted of a total of 1,007,414 cells. The normal cell atlas (NCA) created by the researchers using normal tissue data consisted of a total of 489,575 cells. The extracted data was processed based on the human reference genome (GRCh38) by the researchers.

Finally, an independent database consisting of representative cells for each cell type was established using a sampling method by the researchers. By evenly extracting cells from all over the UMAP coordinate space in which the dimension of data was reduced, 10,000 cells of each cell type were secured by the researchers. The independent database consisted of 367,804 cells, and the top three major constituent cell types included cancer cells (25%), epithelial cells (19%), and fibroblasts (9%).

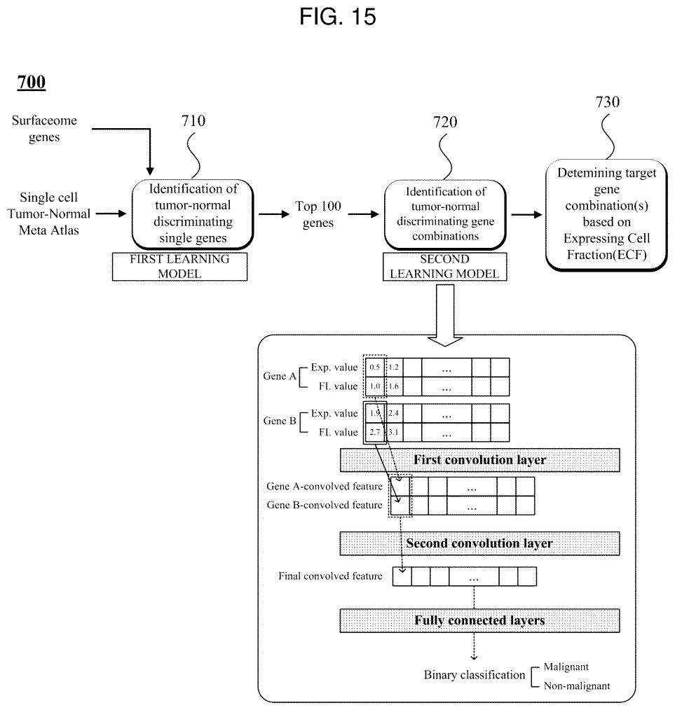

FIG. 15 is an example of the process (700) of identifying a gene combination that discriminates tumor cells and normal cells using a large-scale single cell transcriptome data.

Genes with high expression contribution to discriminating between tumor cells and normal cells among cell surface genes were identified using a learning model (hereinafter, first learning model) by the researchers (710).

The first learning model selected genes with the highest expression contribution to discriminating between tumor cells and normal cells. Random forest was used as a first learning model by the researchers. The model was established using the random forest R package by the researchers. Data for six major types of carcinoma (ovarian cancer (OV), pancreatic adenocarcinoma (PAAD), liver hepatocellular carcinoma (LIHC), non-small cell lung carcinoma (NSCLC), colorectal cancer (CRC), and BRCA-associated breast cancer) derived from epithelial cells were used in the above-described Single cell Tumor-Normal Meta Atlas by the researchers. Accordingly, the first learning model selected genes with high contribution to discriminating between tumor cells and normal cells from the six types of carcinoma.

80% of the established data was used as learning data and the other 20% as verification data by the researchers. The learning data was the Single cell Tumor-Normal Meta Atlas for six types of carcinoma. The leaning data may consist of a specific cell RNA expression data and a label value (tumor or normal) of the corresponding cell. A model built with 100 randomly selected trees and 48 variables was established by the researchers. The first learning model received the specific cell RNA expression data and learned to classify the corresponding cell as a tumor cell or a normal cell.

The performance of the established random forest was evaluated using the verification data by the researchers. A receiver operating characteristic (ROC) of the built model was assessed using the ROCR R package by the researchers. The performance of the established random forest is shown in Table 1 below. Table 1 shows Kappa, accuracy, specificity, sensitivity, error and area under the ROC curve (AUC) for six types of carcinoma. The six types of carcinoma in Table 1 include ovarian cancer (OV), pancreatic adenocarcinoma (PAAD), liver hepatocellular carcinoma (LIHC), non-small cell lung carcinoma (NSCLC), colorectal cancer (CRC), and BRCA-associated breast cancer.

TABLE 1

| Cancer type | OV | PAAD | LIHC | NSCLC | CRC | BRCA |
|---|---|---|---|---|---|---|
| Kappa | 0.951 | 0.961 | 0.941 | 0.946 | 0.976 | 0.950 |
| Accuracy | 0.984 | 0.988 | 0.981 | 0.983 | 0.992 | 0.984 |
| Specificity | 0.995 | 0.995 | 0.986 | 0.997 | 0.999 | 0.993 |
| Sensitivity | 0.944 | 0.960 | 0.962 | 0.928 | 0.967 | 0.949 |
| Error | 0.020 | 0.012 | 0.020 | 0.021 | 0.008 | 0.017 |
| AUC | 0.994 | 0.998 | 0.997 | 0.997 | 0.999 | 0.997 |

The top 100 genes significant in discriminating between tumor cells and normal cells were selected using the first learning model by the researchers (710). The top 100 genes with the highest “average accuracy reduction rate” provided by the random forest R package were selected by the researchers.

A second learning model classified a specific cell as a tumor cell or a normal cell based on the expression pattern of a cell surface gene pair combination in the corresponding cell. A convolutional neural network (CNN) was used as a second learning model by the researchers. The CNN was written in Python 3.8 and constructed with a neural network based on Keras v.2.6.0 and TensorFlow v.2.4.1. CNN learning was conducted based on 5 cross-validation. In addition, consistent results were confirmed by repeating the entire learning process 10 times by the researchers. Learning data consisted of the combinations of the 100 genes determined by the first learning model (random forest) and cell types (normal cells or tumor cells) of the corresponding gene combinations (gene pairs).

Input data of the CNN included single cell RNA-seq expression values and feature-importance values derived from the random forest. The CNN classified a source (cell) of the RNA-seq expression data as a tumor cell or a normal cell based on the corresponding input values.

The structure of the CNN included two convolution layers and fully connected layers. The first convolution layer received information on all possible combinations for the top 100 genes. The first convolution layer of the CNN extracted features per gene combination by combining RNA-seq expression data and feature-importance values for each gene pair (Gene A and Gene B). The second convolution layer of the CNN extracted features by combining the outputs of the first convolution layer for the gene pair (Gene A and Gene B).

The fully connected layers received the features output from the convolution layers and output a probability value for whether a sample being analyzed was normal cells (non-malignant) or tumor cells (malignant).

The model performance of the CNN was evaluated by creating 10 models for each type of carcinoma, and in Table 2, as performance parameters, loss, accuracy, F1 score, precision, recall and AUC are shown.

TABLE 2

| Cancer | Performance | Round1 | Round2 | Round3 | Round4 | Round5 | Round6 | Round7 | Round8 | Round9 | Round10 |
|---|---|---|---|---|---|---|---|---|---|---|---|
| OV | Loss | 0.046 | 0.041 | 0.047 | 0.043 | 0.044 | 0.04 | 0.039 | 0.045 | 0.041 | 0.041 |
| | Accuracy | 0.983 | 0.987 | 0.985 | 0.986 | 0.985 | 0.986 | 0.986 | 0.985 | 0.986 | 0.986 |
| | F1score | 0.983 | 0.987 | 0.985 | 0.986 | 0.985 | 0.986 | 0.986 | 0.985 | 0.986 | 0.986 |
| | Precision | 0.983 | 0.987 | 0.984 | 0.987 | 0.985 | 0.986 | 0.986 | 0.984 | 0.986 | 0.986 |
| | Recall | 0.984 | 0.987 | 0.985 | 0.985 | 0.986 | 0.987 | 0.986 | 0.985 | 0.986 | 0.986 |
| | AUC | 0.999 | 0.999 | 0.999 | 0.999 | 0.999 | 0.999 | 0.999 | 0.999 | 0.999 | 0.999 |
| PAAD | Loss | 0.027 | 0.029 | 0.026 | 0.027 | 0.029 | 0.028 | 0.026 | 0.025 | 0.03 | 0.032 |
| | Accuracy | 0.993 | 0.991 | 0.992 | 0.993 | 0.991 | 0.992 | 0.992 | 0.993 | 0.992 | 0.991 |
| | F1score | 0.992 | 0.991 | 0.992 | 0.992 | 0.991 | 0.992 | 0.993 | 0.993 | 0.992 | 0.991 |
| | Precision | 0.993 | 0.991 | 0.992 | 0.993 | 0.991 | 0.992 | 0.993 | 0.993 | 0.992 | 0.99 |
| | Recall | 0.992 | 0.991 | 0.993 | 0.992 | 0.991 | 0.991 | 0.992 | 0.993 | 0.992 | 0.992 |
| | AUC | 0.999 | 0.999 | 1 | 0.999 | 0.999 | 1 | 0.999 | 1 | 1 | 0.999 |
| LIHC | Loss | 0.037 | 0.031 | 0.043 | 0.046 | 0.046 | 0.035 | 0.046 | 0.04 | 0.039 | 0.042 |
| | Accuracy | 0.984 | 0.988 | 0.983 | 0.982 | 0.98 | 0.984 | 0.981 | 0.982 | 0.984 | 0.982 |
| | F1score | 0.984 | 0.987 | 0.983 | 0.981 | 0.98 | 0.985 | 0.981 | 0.982 | 0.984 | 0.982 |
| | Precision | 0.984 | 0.988 | 0.981 | 0.982 | 0.98 | 0.984 | 0.983 | 0.981 | 0.983 | 0.982 |
| | Recall | 0.984 | 0.987 | 0.985 | 0.981 | 0.98 | 0.985 | 0.979 | 0.982 | 0.985 | 0.982 |
| | AUC | 0.999 | 0.999 | 0.999 | 0.999 | 0.999 | 1 | 0.999 | 0.999 | 0.999 | 0.999 |
| NSCLC | Loss | 0.039 | 0.04 | 0.041 | 0.036 | 0.038 | 0.034 | 0.036 | 0.044 | 0.052 | 0.038 |
| | Accuracy | 0.988 | 0.986 | 0.984 | 0.988 | 0.986 | 0.988 | 0.987 | 0.986 | 0.983 | 0.986 |
| | F1score | 0.988 | 0.985 | 0.985 | 0.988 | 0.986 | 0.988 | 0.987 | 0.986 | 0.984 | 0.986 |
| | Precision | 0.987 | 0.986 | 0.984 | 0.988 | 0.986 | 0.988 | 0.987 | 0.985 | 0.983 | 0.986 |
| | Recall | 0.988 | 0.985 | 0.985 | 0.987 | 0.986 | 0.988 | 0.987 | 0.986 | 0.984 | 0.986 |
| | AUC | 0.999 | 0.999 | 0.999 | 0.999 | 0.999 | 1 | 0.999 | 0.999 | 0.999 | 0.999 |
| CRC | Loss | 0.017 | 0.017 | 0.015 | 0.014 | 0.013 | 0.018 | 0.015 | 0.015 | 0.017 | 0.012 |
| | Accuracy | 0.995 | 0.995 | 0.995 | 0.996 | 0.996 | 0.995 | 0.995 | 0.996 | 0.996 | 0.996 |
| | F1score | 0.995 | 0.995 | 0.995 | 0.996 | 0.996 | 0.995 | 0.995 | 0.996 | 0.996 | 0.996 |
| | Precision | 0.995 | 0.995 | 0.996 | 0.996 | 0.997 | 0.995 | 0.995 | 0.996 | 0.996 | 0.996 |
| | Recall | 0.995 | 0.995 | 0.995 | 0.996 | 0.996 | 0.995 | 0.995 | 0.996 | 0.996 | 0.996 |
| | AUC | 0.799 | 0.792 | 0.854 | 0.779 | 0.813 | 0.862 | 0.77 | 0.839 | 0.815 | 0.786 |
| BRCA | Loss | 0.038 | 0.041 | 0.044 | 0.045 | 0.048 | 0.041 | 0.046 | 0.04 | 0.045 | 0.049 |
| | Accuracy | 0.987 | 0.987 | 0.985 | 0.986 | 0.983 | 0.987 | 0.985 | 0.987 | 0.986 | 0.984 |
| | F1score | 0.987 | 0.987 | 0.985 | 0.986 | 0.983 | 0.98 | 0.985 | 0.987 | 0.98 | 0.984 |
| | Precision | 0.987 | 0.987 | 0.986 | 0.987 | 0.984 | 0.987 | 0.985 | 0.987 | 0.986 | 0.984 |
| | Recall | 0.987 | 0.987 | 0.985 | 0.985 | 0.983 | 0.987 | 0.985 | 0.987 | 0.986 | 0.984 |
| | AUC | 0.999 | 0.999 | 0.999 | 0.999 | 0.998 | 0.999 | 0.998 | 0.999 | 0.999 | 0.998 |

Input data significant in CNN classification was confirmed by the researchers. A visualization configuration (Grad-CAM) was realized to visually confirm feature values significant in classification by the researchers. In the end, the top significant gene combinations or the ranking of each gene combination may be confirmed through CNN classifying tumor cells and normal cells by the researchers.

Afterward, the final target gene combination(s) were selected based on expressing cell fraction (ECF) by the researchers (730).

First, for each cell and each gene, expression values from single-cell RNA-seq data were converted into a binary format (0 or 1) representing “expressed or not.” The ECF of each gene was defined as the percentage of cells, indicating the expression of the corresponding gene in the defined corresponding group (tumor cell group or normal cell group).

Based on ECFs of tumor cells and normal cells, the final gene combination(s) for a target antigen were determined from the gene combinations. In order to calculate ECF per logic circuit (AND, OR, or NOT) for gene pair combinations, it was checked whether gene combinations for each cell were expressed in tumor cells or normal cells. To identify whether expression occurred in specific cells according to AND logic, the case in which two genes were expressed simultaneously was set as “expressed.” To identify whether expression occurred in specific cells according to OR logic, the case in which one of the two genes or both genes were expressed was set as “expressed.” To identify whether expression occurred in specific cells according to NOT logic, the case in which genes in the front order of a gene pair combination were expressed and genes in the back order of the combination were not expressed was set as “expressed” for the corresponding combination. The expression of a gene combination was determined according to the logic circuit as described above, and it was expressed again in the percentages of cells exhibiting gene combination expression. From the gene combinations identified by the CNN, the top 25% combinations were selected based on a CNN learning contribution, and from the top 25% combinations, combinations showing 75% or more ECF in a tumor cell population and less than 10% ECF in a normal cell population were selected.

The final gene combination extracted from the constructed data set was verified based on the actual protein expression pattern by the researchers. Based on CITE-seq data acquired using ovarian cancer and colorectal cancer tissue samples, single cell genome expression data and surface protein expression data were pre-processed constantly by the researchers, and compared by cell type using tSNE coordinates.

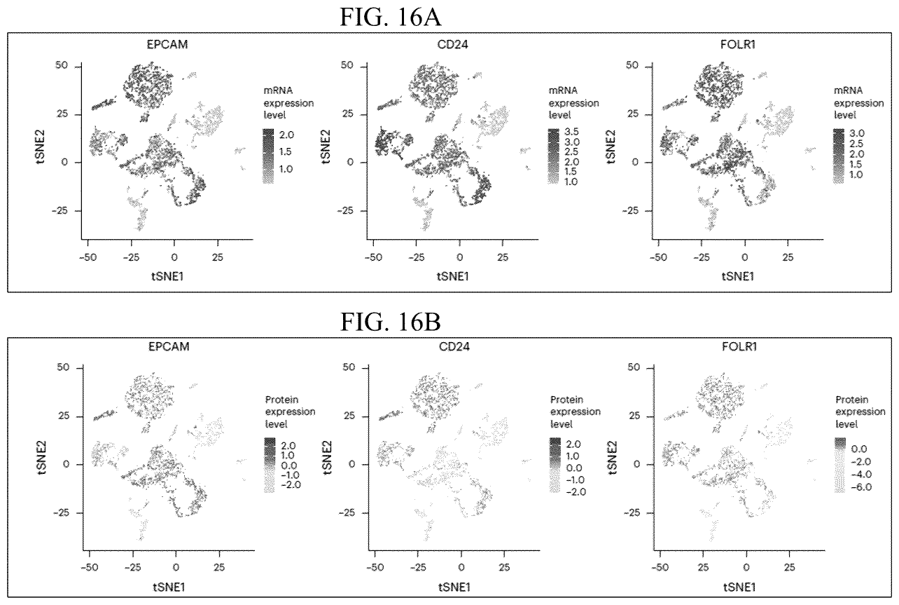

FIGS. 16A and 16B illustrate a result of comparing patterns of RNA expression and protein expression against an antigen of ovarian cancer cells. FIG. 16 is an example of analyzing EPCAM, CD24 and FOLR1 among known antigens. FIG. 16A shows the expression pattern of RNA in tSNE coordinates. FIG. 16A is the expression pattern for the final gene combination determined in the process of FIG. 15. Referring to FIG. 16, it can be seen that the gene combination determined by a learning model is significant for discovering an actual antigen by the researchers.

FIGS. 3 to 6 described above illustrate the target antigen discovery processes for a CAR. In addition, FIG. 15 illustrates the process of extracting a gene combination for a candidate antigen using a learning model. The analysis apparatus may discover a target antigen for a CAR using the automated gene combination extraction process described in FIG. 15.

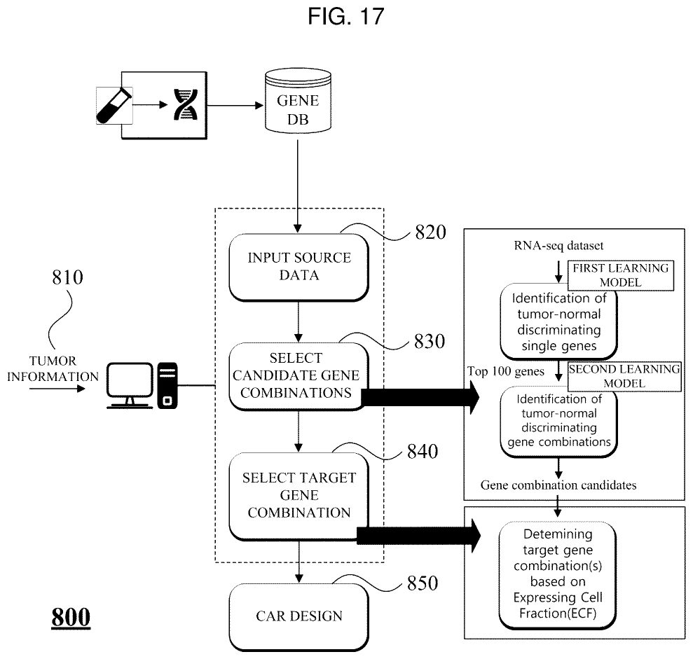

FIG. 17 is another example of a process (800) of discovering a target antigen for a CAR.

A gene DB stores gene expression data of tumor cells. The gene DB may store gene expression information of normal cells. In addition, the gene DB may store information on the correlation in expression between genes. The gene DB may store RNA expression data for tumor cells and normal cells. The gene DB may be a public database or an individual database established by a developer.

The analysis apparatus may receive tumor information to be analyzed (810). The tumor information may include the type of tumor, clinical information, patient information, etc. (i) The tumor information may define a specific tumor under a specific clinical condition. The clinical information may include the ethnicity, sex, and age of a sample, a stage of tumor progression (early, intermediate, terminal), and an anatomical area from which a sample is extracted. In this case, the tumor information is used to define a specific tumor having specific clinical conditions. (ii) The tumor information may also be used to define a specific tumor for a specific patient.

The analysis apparatus may input or receive source data corresponding to tumor information input by the gene DB (820). The source data may consist of RNA expression data of tumor cells corresponding to tumor information (based on at least one or more of tumor type, ethnicity, sex, and tissue) and normal cells with respect to the corresponding manipulation.

The analysis apparatus may select candidate gene combinations from the source data (830). Here, the analysis apparatus may select the candidate gene combinations using a learning model. A learning model may be a model established by any one method of decision trees, random forest (RF), K-nearest neighbor (KNN), Naive Bayes, a support vector machine (SVM), deep neural network (DNN), and a regression model.

The analysis apparatus may select a gene with a high expression contribution to discriminate between normal cells and tumor cells from the source data using a learning model. For example, the analysis apparatus may select single genes significant in discriminating between normal cells and tumor cells from an RNA-seq data set input using the first learning model illustrated in FIG. 15. For example, the analysis apparatus may select the top 100 single genes.

The analysis apparatus may select a gene combination by a contribution to discriminating between normal cells and tumor cells from the source data using a learning model. For example, the analysis apparatus may select the top-rated gene combinations having a high contribution to discriminating between normal cells and tumor cells from all possible combinations for genes input by the second learning model illustrated in FIG. 15. The second learning model produces contribution information on all gene combinations by receiving feature-importance values produced by RNA-seq expression data for gene pairs and the first learning model. The analysis apparatus may select top-rated gene combinations based on the ranking of gene combinations confirmed in the analysis process by the second learning model. For example, the analysis apparatus may select gene combinations having a contribution of the reference value or more, or the top few % of gene combinations of all of the gene combinations as candidate gene combinations.

The analysis apparatus may select a target gene combination based on the coverage of a gene included in each of the candidate gene combinations (840). The analysis apparatus may select the final target gene combination(s) based on ECF. For example, (i) when genes included in a gene combination are expressed at the reference percentage (e.g., 60%) or more in a tumor cell population, the analysis apparatus may select the corresponding gene combination as a target gene combination. In addition, (ii) when genes included in a gene combination are expressed at less than the reference percentage (e.g., 10%) in a normal cell population, the analysis apparatus may select the corresponding gene combination as a target gene combination. In addition, (iii) when genes included in a gene combination are expressed at the first reference percentage (e.g., 60%) or more in a tumor cell population and at less than the second reference percentage (e.g., 10%) in a normal cell population, the analysis apparatus may select the corresponding gene combination as a target gene combination.

Afterward, the analysis apparatus may deliver the final target antigen information for a CAR to another object. Alternatively, the analysis apparatus may also design a recognition site for a CAR, which recognizes the final target antigen (850).

An analysis apparatus 600 that discovers a target antigen for a CAR will be explained. As described above, the analysis apparatus 600 is an apparatus corresponding to the analysis apparatus 130 or 140 of FIG. 1. The content overlapping that described in FIG. 14 will not be described.

The storage device 610 may store a learning model for identifying genes and gene combinations, significant in discriminating between normal cells and tumor cells. For example, the storage device 610 may store the above-described first learning model and second learning model.

The storage device 610 may store RNA expression data in tumor cells and normal cells for various populations. A tumor may be at least one of various types of tumors. That is, the storage device 610 may store the gene DB of FIG. 17.

The storage device 610 stores a program that determines an antigen recognized by a CAR using the expression information of genes in tumor cells.

The memory 620 may store data and information produced in the process of analyzing genetic data by the analysis apparatus 600.

The interface device 640 is an apparatus that receives certain commands and data from the outside. The interface device 640 may receive RNA expression data from a physically connected input device or external storage device. The interface device 640 may receive RNA expression data in tumor cells and normal cells of a sample in a single cell unit. The interface device 640 may also receive tumor information for analysis.

The communication device 650 is a component that receives and transmits certain information via wired or wireless network. The communication device 650 may receive RNA expression data from an external object. The communication device 650 may receive RNA expression data in tumor cells and normal cells of a sample in a single cell unit. The communication device 650 may receive tumor information for analysis. The communication device 650 may transmit significant gene information or target antigen-associated information, which is an analysis result.

The interface device 640 may be an apparatus that delivers data received from the communication device 650 into the analysis apparatus 600.

The output device 660 is an apparatus that outputs certain information. The output device 660 may output an interface, an analysis result, etc., necessary for a data treatment process.

The arithmetic device 630 may discover a target antigen by analyzing genetic data using a program stored in the storage device 610.

The arithmetic device 630 may select single genes significant in discriminating between normal cells and tumor cells from an RNA-seq data set input using the first learning model. For example, the analysis apparatus may select the top 100 single genes.

The arithmetic device 630 may select top-rated gene combinations having a high contribution to discriminating between normal cells and tumor cells from all possible combinations for genes input using the second learning model. The second learning model produces contribution information on all gene combinations by receiving RNA-seq expression data for gene pairs and the feature-importance values produced by the first learning model. The arithmetic device 630 may select top-rated gene combinations based on the ranking of gene combinations confirmed in the analysis process of the second learning model. The arithmetic device 630 may select gene combinations having a contribution of the reference value or more or the top-rated few % of gene combinations of all gene combinations as candidate gene combinations.

The arithmetic device 630 may select a target gene combination based on the coverage of a gene included in each of the candidate gene combinations. For example, when genes included in a gene combination are expressed at the first reference percentage (e.g., 60%) or more in a tumor cell population and at less than the second reference percentage (e.g., 10%) in a normal cell population, the arithmetic device 630 may select the corresponding gene combination as a target gene combination.

The arithmetic device 630 may use a leaning model in the process of determining a gene combination based on the positive candidate genes and the negative candidate genes in FIG. 5. The arithmetic device 630 may select genes of candidate antigens using a first learning model (420). In addition, the arithmetic device 630 may select top-rated gene combinations from gene combinations consisting of positive candidate genes and negative candidate genes using a second learning model. Afterward, the arithmetic device 630 may determine a target gene combination according to expression percentages in normal cells and tumor cells for each gene combination.

The arithmetic device 630 may be an apparatus for processing data and certain calculations, such as a processor, AP, or a program-embedded chip.

In addition, the target antigen discovery method for a CAR as described above may be implemented by a program (or application) including an executed algorithm that can be run by a computer. The program may be provided by being stored in a transitory or non-transitory computer readable medium.

The non-transitory computer readable medium is a medium that stores data semi-permanently and is able to be read by an apparatus, rather than a medium that stores data temporarily, such as a register, a cache, or a memory. Specifically, the above-described various applications or programs may be provided by being stored in a non-transitory computer readable medium such as a CD, DVD, hard disk, Blu-ray disc, USB, memory card, read-only memory (ROM), programmable read-only memory (PROM), erasable PROM (EPROM), electrically erasable PROM (EEPROM), or flash memory.

The transitory computer readable medium includes various RAMs, such as a static RAM (RAM), dynamic RAM (DRAM), synchronous DRAM (SDRAM), double data Rate SDRAM (DDR SDRAM), enhanced SDRAM (ESDRAM), Synclink DRAM (SLDRAM), and direct Rambus RAM (DRRAM).

The present embodiments and the accompanying drawings in the specification merely clearly show parts of the technical ideas included in the above-described technology, and it should be apparent that all modifications and specific examples that can be easily inferred by those of ordinary skill in the art included in the specification and drawings of the above-described technology are included in the scope of rights of the above-described technology.

What is claimed is:

1. A target antigen discovery method for a chimeric antigen receptor (CAR), comprising:
acquiring expression information of genes in tumor cells in a single cell unit by an analysis apparatus;
extracting genes of candidate antigens having an expression level of a reference value or higher from the genes by the analysis apparatus;
determining gene combinations which can consist of the genes of the candidate antigens by the analysis apparatus;
determining a target gene combination in which at least one gene included in the combination is expressed among the gene combinations by the analysis apparatus; and
determining at least one of the target gene compositions as a gene combination for a target antigen by the analysis apparatus,
wherein the at least one gene is a gene for an antigen in the tumor cells, recognized by the CAR, and the target gene combination is a gene combination in which the percentage of cells in which the at least one gene is expressed in tumor cells is the reference value or more.

2. The method of claim 1, wherein the analysis apparatus extracts genes in which an expressing percentage in the tumor cells is the reference value or more among genes of the tumor cells as genes of the candidate antigens.

3. The method of claim 1, wherein the analysis apparatus extracts genes in which a difference in expression level in normal cells and the tumor cells is the reference value or more among genes of the tumor cells as genes of the candidate antigens.

4. The method of claim 1, wherein the analysis apparatus determines the target gene combination in which a plurality of genes comprised therein are expressed from the gene combinations in which the target gene combination is a gene combination in which the percentage of cells in which the plurality of genes are simultaneously expressed among the tumor cells is the reference value or more.

5. The method of claim 4, wherein the plurality of genes are involved in expression of different antigens.

6. The method of claim 1, wherein the analysis apparatus extracts genes of the candidate antigens significant in discriminating between normal cells and tumor cells among the genes using a first learning model.

7. The method of claim 1, wherein the analysis apparatus determines the target gene combination significant in discriminating between cells in the gene combinations and tumor cells using a second learning model.

8. A target antigen discovery method for a chimeric antigen receptor (CAR), comprising:
acquiring expression information of genes of tumor cells in a single cell unit by an analysis apparatus;
extracting genes of candidate antigens having an expression level of a reference value or higher from the genes by the analysis apparatus;
determining positive candidate genes from genes of the candidate antigens by the analysis apparatus, based on the percentage of expressing cells in the tumor cells or the number of expressing cells in the tumor cells;
determining negative candidate genes whose expression pattern has a reverse relationship with the positive candidate genes among genes of the candidate antigens by the analysis apparatus;
determining gene combinations that can be composed of the positive candidate genes and the negative candidate genes by the analysis apparatus;
determining a target gene combination for an antigen recognized by the CAR among the gene combinations by the analysis apparatus, based on the percentage of cells in which at least one positive candidate gene included in a combination of the gene combinations is expressed among the tumor cells and the percentage of cells in which at least one negative candidate gene of the negative candidate genes inhibiting an immune response of CAR cells for an antigen produced by expressing the at least one positive candidate gene is expressed among normal cells; and
determining at least one combination of the target gene combinations as a gene combination for a target antigen by the analysis apparatus.

9. The method of claim 8, wherein the analysis apparatus extracts genes in which the percentage of expressing cells in the tumor cells is the reference value or more among genes of the tumor cells as genes of the candidate antigens.

10. The method of claim 8, wherein the analysis apparatus extracts genes in which a difference in expression level in normal cells and the tumor cells is the reference value or more among genes of the tumor cells as genes of the candidate antigens.

11. The method of claim 8, wherein the analysis apparatus determines the specific combination in which the percentage of tumor cells in which at least one positive candidate gene of the positive candidate genes included in a specific combination is expressed among the tumor cells is the reference value or more, and
the percentage of normal cells in which a suppressive gene suppressing immune responses of an antigen and the CAR by the positive candidate gene is expressed among normal cells in which at least one positive candidate gene is expressed is the reference value or more as the target gene combination.

12. The method of claim 8, wherein the positive candidate genes are involved in expression of different antigens.

13. An analysis apparatus for designing a chimeric antigen receptor (CAR), comprising:
an input device for receiving expression data of genes in tumor cells of a sample in a single cell unit;
a storage device for storing a program that determines an antigen recognized by the CAR using the expression information of genes in tumor cells; and
an arithmetic device for determining a target gene combination or target gene for a target antigen of tumor cells in the sample recognized by the CAR from gene combinations that can consist of a plurality of genes among the genes of the candidate antigens determined from the expression data of the genes,
wherein the target gene combination has the percentage of cells in which at least one gene included therein is expressed among the tumor cells of the reference value or more.

14. The analysis apparatus of claim 13, wherein genes of the candidate antigens are genes in which the percentage of expressing cells in the tumor cells is a first reference value or more among genes of the tumor cells.

15. The analysis apparatus of claim 13, wherein genes of the candidate antigens are genes in which a difference in expression level in normal cells and the tumor cells is the reference value or more among genes of the tumor cells.

16. The analysis apparatus of claim 14, wherein the plurality of genes have the percentage of expressing cells in the tumor cells of a second reference value or more among genes of the candidate antigens, and the second reference value is a value higher than the first reference value.

17. The analysis apparatus of claim 13, wherein the target gene combination has the percentage of cells in which at least a plurality of genes are expressed included in the target gene combination among the tumor cells of the reference value or more.

18. The analysis apparatus of claim 13, wherein the input device further receives expression data of genes of normal cells of the sample,
the arithmetic device determines positive candidate genes and negative candidate genes whose expression pattern has a reverse relationship with the positive candidate genes from genes of the candidate antigens, based on the percentage of expressing cells in tumor cells or the number of expressing cells in the tumor cells, and
all gene combinations that are able to be composed of the positive candidate genes and the negative candidate genes are determined as the gene combinations.

19. The analysis apparatus of claim 18, wherein the arithmetic device determines a specific combination in which the percentage of cells in which at least one positive candidate gene among the positive candidate genes included in the specific combination is expressed among the tumor cells is the reference value or more, and
the percentage of normal cells in which a suppressive gene suppressing recognition or binding of an antigen or the CAR, involving the positive candidate gene, among normal cells in which the at least one positive candidate gene is expressed is the reference value or more as the target gene combination.

20. The method of claim 13, wherein the arithmetic device extracts genes of the candidate antigens significant in discriminating between normal cells and tumor cells among the genes using a first learning model, and determines the target gene combination significant in discriminating between cells in the gene combinations and tumor cells using a second learning model.
